(19) United States
(12) Patent Application Publication
(10) Pub. No.: US 2007/0016249 A1
(43) Pub. Date: Jan. 18, 2007

(54) Suture device

(76) Inventor: Alan M. Reznik, Woodbridge, CT (US)

Correspondence Address: Christopher J. McDonald;HOFFMAN, WASSON & GITLER P.C., Suite 522, 2461 South Clark Street, Arlington, VA, 22202, US

(21) Appl. No.: 11/415,308

(22) Filed: May 2, 2006

Related U.S. Application Data
(60) Provisional application No. 60/698,960, filed on Jul. 14, 2005.

(51) Int. Cl. A61B 17/00

(52) U.S. Cl. 606/207

(57) ABSTRACT

Forceps have handles for manipulating two jaws connected to one another at a hinge point. Extending from the hinge point is the first section serving as a suture grasper. Following the first section is a second section forming a suture passer. When closed, the jaws of the first section come into contact, or close proximity, so as to be able to firmly grasp a suture. The second section has jaws which are spaced from one another to form a suture passer. The distal ends of the jaws forming the second section may come into contact to form a second gripping area.

This application claims benefit of provisional application 60/698,960, filed Jul. 14, 2005.

### BACKGROUND OF THE INVENTION

During surgeries, surgeons use forceps to manipulate tissue and sutures. Different aspects of an operation require different movements of the tissue and sutures, sometimes requiring different forceps. Changing forceps during the course of an operation is undesirable and trying to use a single forceps to perform different tasks is cumbersome. There is a need in the prior art for forceps capable of performing different functions so that the surgeon may use one forceps during the entire course of an operation.

It is an object of the invention to provide a pair of forceps capable of performing many functions.

It is another object of the invention to provide a pair of forceps which a surgeon may use for multiple aspects of an operation.

It is another object of the invention to provide a pair of multi-functional forceps that are convenient to use.

These an other objects of the invention will become apparent to one of ordinary skill in the art after reading the disclosure of the invention.

### SUMMARY OF THE INVENTION

Forceps have handles for manipulating two jaws connected to one another at a hinge point. Extending from the hinge point is the first section serving as a suture grasper. Following the first section is a second section forming a suture passer. When closed, the jaws of the first section come into contact, or close proximity, so as to be able to firmly grasp a suture. The second section has jaws which are spaced from one another to form a suture passer. The distal ends of the jaws forming the second section may come into contact to form a second gripping area.

### BRIEF DESCRIPTION OF THE DRAWINGS

FIG. 1 is a view of the forceps;

FIG. 2 is a view of the jaws of the forceps in the closed position;

FIG. 3 is a view of the jaws in the open position; and

FIG. 4 is a top view of the upper jaw.

### DETAILED DESCRIPTION OF THE INVENTION

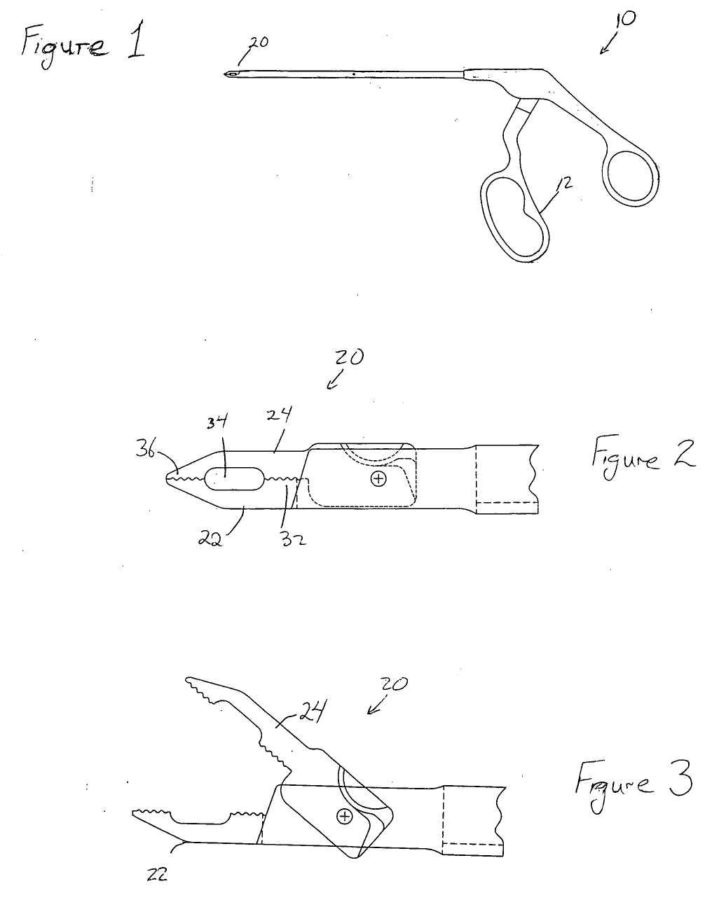

A forceps 10 are seen in FIG. 1. The forceps have a handle 12 and a pair of jaws 20. Manipulation of the handles 12 open and close the jaws, as is well known and conventional in the art. The mechanism for transferring motion of the handles to motion of the jaws is well known.

FIG. 2 shows the details of the jaws 20. The jaws have a lower jaw 22 and an upper jaw 24 connected to one another at a hinge point. The jaws 20 have a first section 32 closest to the hinge point. In the closed position, the jaws come in contact in the first section 32 and form a suture grasper. Next to the first section is a second section 34 where the upper and lower jaw do not touch. This second section forms a suture passer. The suture passer may have any size but preferable has a length of 2.9 mm and a width of 1 mm when the jaws are closed, the width being formed equally by the upper and lower jaw. The third section 36 is formed at the tip of the jaws. Like the first section, the jaws come in contact in the third section to form a suture grasper. Being further from the hinge point, the third section has more movement during twisting and pivoting of the forceps as compared to the first section. However, the first section has a tighter grip on the sutures placed between the jaws allowing the surgeon to tug and pull with greater force without the suture becoming free of the jaws.

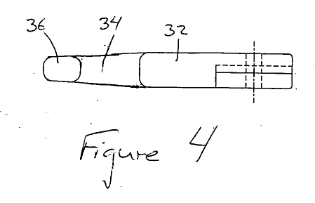

FIG. 3 shows the open position of the jaws with the upper jaw pivoting relative to the lower jaw. It is possible to have a set of jaws where both the upper and lower jaws pivot relative to the main shaft of the forceps. Opening of the jaws allows the sutures to be placed between the jaws in the first or third section so that, when upon closing, the suture is securely grasped by the jaws and manipulated by the surgeon.

While the surface of the first and third sections are shown as being serrated, either section or both may be smooth. Also, the tip of the jaws may be angled or curved and even pointed as best suited for the particular operation forceps will be used.

FIG. 4 depicts the rounded corners of the first section 32 and second section 36. The rounded corners facilitate the passage of a suture into and through the second section 34. The corners of the lower jaw are likewise rounded.

While the invention has been described with reference to a preferred embodiment, variation and modifications would be apparent to one of ordinary skill in the art. The invention encompasses such variations and modifications.

I claim:

1. A forceps, comprising
a) a handle;
b) a shaft extending from said handle;
c) a pair of jaws attached to said handle, said pair of jaws comprising;
d) a lower jaw and an upper jaw, said upper and lower jaw connected to one another at a hinge;
e) a first section, the upper and lower jaw coming into contact when the jaws are in a closed position; and
f) a second section next to said first section, the upper and lower jaws spaced from one another in the second section when the jaws are in the closed position.

2. The forceps of claim 1, wherein said first section is closer to the hinge than said second section.

3. The forceps of claim 1, further comprising a third section.

4. The forceps of claim 1, wherein the upper and lower jaws are serrated in said first section.

5. The forceps of claim 3, wherein the upper and lower jaws are serrated in said third section.

6. The forceps of claim 1, wherein said jaws are smooth in said second section.

7. The forceps of claim 1, wherein corners of the first and third section are rounded.
